(19) United States
(12) Patent Application Publication
(10) Pub. No.: US 2023/0270349 A1
(43) Pub. Date: Aug. 31, 2023

(54) MAGNETIC RESONANCE IMAGING

(71) Applicant: Oxford University Innovation Limited, Oxford (GB)

(72) Inventors: Elizabeth Mary TUNNICLIFFE, Oxford (Headington) (GB); Aaron Timothy HESS, Oxford (Headington) (GB); Betty RAMAN, Oxford (Headington) (GB)

(21) Appl. No.: 18/143,817

(22) Filed: May 5, 2023

Related U.S. Application Data
(62) Division of application No. 16/674,104, filed on Nov. 5, 2019.

(30) Foreign Application Priority Data
Nov. 7, 2018 (GB) 1818147.9

(51) Int. Cl. A61B 5/055; G01R 33/34; G01R 33/385; G01R 33/48

(52) U.S. Cl. CPC A61B 5/055; G01R 33/34; G01R 33/385; G01R 33/4806; A61B 2090/374

(57) ABSTRACT

The present invention relates generally to medical imaging and, more particularly, relates to systems and methods for obtaining magnetic resonance (MR) images of tissues and organs (particularly of the heart) or parts thereof.

### CROSS-REFERENCE TO RELATED APPLICATION

This application claims priority to GB 1818147.9, filed Nov. 7, 2018, which is entirely incorporated herein by reference.

### TECHNICAL FIELD

The present invention relates generally to medical imaging and, more particularly, relates to systems and methods for obtaining magnetic resonance (MR) images of tissues and organs (particularly of the heart) or parts thereof.

### BACKGROUND

In magnetic resonance (MR) imaging, tissue contrast is generated by a combination of intrinsic tissue properties such as spin-lattice (T1) and spin-spin (T2) relaxation times, and extrinsic properties such as imaging strategies and settings. Signal intensity in conventional MR images is displayed on an arbitrary scale, and thus is not adequate for direct comparisons between subjects.

Blood oxygen level dependent (BOLD) imaging harnesses the paramagnetic property of deoxyhaemoglobin to non-invasively assess tissue oxygenation (1). Haemoglobin has different magnetic properties in its oxygenated and deoxygenated forms: deoxygenated haemoglobin is paramagnetic and oxygenated haemoglobin is diamagnetic. Both contribute to the signal detected using magnetic resonance imaging (MRI) and variations in the ratio between oxygenated and deoxygenated haemoglobin lead to signal variations which can be detected using an MRI scanner. BOLD imaging is usually carried out at rest and then under the action of a vasodilator stress such as adenosine. In healthy tissue this leads to an increase in blood flow and reduction in deoxyhaemoglobin, which in turn is accompanied by an increase in signal intensity. In diseases, where there is a narrowing in the blood vessels supplying tissue, the blood flow to biological tissue is reduced resulting in in blunting of change in deoxyhaemoglobin and blunted BOLD response. Heavily T2* weighted sequences are often used to detect these variations, which are in the order of 1-5%.

Previous studies have successfully applied BOLD to understand the relationship between myocardial blood flow and tissue oxygenation in cardiovascular diseases (2, 3). Stress BOLD was recently shown to accurately detect functionally-relevant flow-limiting coronary stenosis without the need for extrinsic contrast agents, addressing a critical limitation of current non-invasive diagnostic techniques for the direct assessment of ischaemia (4). In a pivotal study, Vöhringer et al. (5) demonstrated that the change in contrast on SSFP cine-BOLD induced by vasoactive substances strongly associates with myocardial oxygenation rather than blood flow. This suggests that BOLD is sensitive to the physiological effects of increased blood flow unlike contrast enhanced perfusion assessment methods on CMR.

Early myocardial BOLD studies used either T2*-weighted images (6) or T2* mapping (7), but these techniques suffered from relatively low signal-to-noise ratio (SNR) and artefacts caused by magnetic field inhomogeneity and motion. More recently, the field has moved towards SSFP-based methods. These include long-TR SSFP cine (5), using the native T2 sensitivity of steady state balanced SSFP, or using a T2 preparation module with an SSFP readout (8).

Despite the promising nature of myocardial BOLD imaging, all previous techniques have shown wide normal ranges, with population standard deviations comparable in size to the mean BOLD change on adenosine stress. For example, BOLD T2* changes of 17±9% (7), cine signal changes of 3.9±6.5% (9), and T2-prepared SSFP signal changes of 12±11% (10) to 20±7% have been found (11). This limits the sensitivity and specificity of the technique in detecting disease which has, in turn, limits the regional or segmental assessment of tissue oxygenation using the technique. For example, in the study by Arnold et al. (10), a segmental analysis failed to identify regions affected by critical flow limiting stenosis. While the cine SSFP method relies on a true steady state of the magnetization and thus has no heart-rate dependence and mapping methods are also heart-rate independent, T2*-weighted or T2-weighted methods usually rely on some kind of heart-rate correction to account for the change in steady state longitudinal magnetization during stress imaging (6, 12).

Heart rate correction is required in T2-weighted BOLD imaging, as in some T2*-weighted methods (6), because there is insufficient time during a breath-hold for full T1 recovery between multiple T2 preparation pulses. As a result, the signal in the SSFP readouts is sensitive to the subject's heart rate as well as the T2 of the myocardium. Heart rate correction aims to remove this effect. Existing methods are imperfect because they assume that the tissue relaxation parameters are the same between rest and stress.

There is therefore a need for further MRI methods which provide at least some degree of heart-rate compensation or correction when used in myocardial BOLD imaging.

Kellman et al. (13) reported normalisation of a T2-prepared SSFP sequence for imaging myocardial oedema. This normalisation was used to manage surface coil sensitivity variations by interleaving low-flip angle FLASH reference images between the SSFP readouts. The SSFP images were normalized by the FLASH images, and the correction of signal intensity variation across the image enhanced the visibility of subtle changes in signal intensity due to myocardial oedema.

The same normalisation method was used by Yang et al. (24). However, the authors of this paper made no mention of any need for heart-rate correction in T2-prepared SSFP BOLD and they did not disclose the specific method that they used. From the wide ranges in BOLD signals in all groups where a statistically-significant heart rate change was measured, it can be inferred that no heart-rate compensation was attempted by Yang et al.

### SUMMARY

It has now been found that normalisation of T2 prepared SSFP-BOLD images by interleaved FLASH images considerably reduces both segmental and individual variability of the derived BOLD changes in signal intensity without altering the magnitude of the BOLD effect measured. In particular, it has been found that the normalization of the SSFP images by the FLASH images accounts for changes in steady state longitudinal magnetization due to changing heart rate and saturation by the SSFP readout train, therefore providing more accurate heart rate correction than previously available.

It is therefore an object of the invention to provide a method of obtaining a heart-rate compensated magnetic resonance (MR) image of all or part of a tissue or organ.

In one embodiment, therefore, the invention provides a computer-implemented method for obtaining a heart-rate-compensated magnetic resonance (MR) image of all or part of a tissue or organ of a subject, the method comprising the steps:
(a) acquiring, with an MR system, an MR data set from all or part of a tissue or organ of a subject using a pulse sequence, wherein the pulse sequence comprises at least two interleaved components:
   (i) a first component, wherein the first component consists of a T2- or T2*-weighted readout, and
   (ii) a second component, wherein the second component is a low flip angle readout without additional magnetisation preparation;
(b) generating at least two image datasets from the MR dataset, a first image dataset derived from the signals obtained from the first component of the pulse sequence, and a second image dataset derived from the signals obtained from the second component of the pulse sequence;
(c) normalising the first image dataset using the second image dataset as a reference dataset to produce a third image dataset; and optionally;
(d) displaying, from the third image data set, a heart-rate-compensated MR image of all or part of the tissue or organ.

The method of the invention is computer-implemented. For example, the method may be implemented on a computerised system having a processor and non-transitory computer medium. This may be operatively connected to an MRI scanner. The scanner may have an MR data acquisition unit which is capable of acquiring MR data, e.g. from a predetermined volume of the subject.

In some embodiments, the method of the invention does not require the subject to hold his/her breath during MR data acquisition, i.e. the method is a non-breath-hold method. In some embodiments, the method of the invention requires the subject to hold his/her breath during MR data acquisition, i.e. the method is a breath-hold method.

The method of the invention is for obtaining a heart-rate compensated magnetic resonance (MR) image. As used herein, the term “heart-rate compensated image” means that the variability of one or both of the intra-subject segmental and inter-subject averaged signal intensities of the MR images are reduced compared to the variability found in a control non-normalised image. The method used also effectively compensates for differences in signal due to surface coil sensitivity variations within and between different subjects.

The term “heart-rate compensated MR image” may also mean that variability caused by changes in the steady state longitudinal magnetization due to changing heart rate, which occurs during vasodilator stress, in the subject is reduced in the compensated MR image. Preferably, the variability is reduced without altering the magnitude of the determined BOLD signal intensity change between rest and stress. Preferably the reduction in variability in signal in the heart-rate compensated MR image also leads to a reduction in inter-subject variability in BOLD signal intensity change between rest and stress. Preferably, the variability in image signal intensity is reduced such that the normal ranges of signal intensities measured in healthy volunteers at rest and stress do not overlap when the normal range is determined by calculating the mean plus or minus twice the standard deviation of the signal intensity.

The method provides an MR image of all or part of a tissue or organ of a subject. The subject may be any animal, preferably a mammal, most preferably a human. The subject is preferably alive, i.e. having a heart-beat and a heart-rate.

The method provides an MR image of all or part of a tissue or organ of the subject. The tissue or organ may be any biological tissue or organ with a vascular bed, preferably one which is capable of reacting to external and/or internal vasoactive (e.g. vasodilatory or vasoconstrictive) stimuli. Preferably, the organ is a visceral organ, e.g. a heart, liver, spleen, kidney, prostate, lung or pancreas. In other embodiments, the tissue or organ is the brain or a muscle.

Preferably, the organ is a heart, most preferably a human heart. In some embodiments, the tissue or organ is the myocardium. Preferably, the tissue or organ is the left ventricular myocardium. In other embodiments, the tissue or organ is the whole heart or a slice thereof.

The MRI measurements are taken in a Region Of Interest (ROI) which may be automatically segmented, on a pixel-pixel basis, or chosen as a ROI by the operator.

In some embodiments, the tissue or organ is impaired or diseased. For example, the tissue or organ may be one which has reduced oxygenation and/or blood flow compared to a normal, healthy (reference) tissue or organ. In some embodiments, the impairment in oxygenation and/or blood flow is induced artificially, i.e. by a chemical or physical stimulus (e.g. by a vasodilatory or vasoconstrictive agent). In other embodiments, the reduced oxygenation and/or blood flow is due to a disease or disorder, e.g. a coronary disease, a cardiomyopathy due to a genetic, metabolic or structural (e.g. valvular) disorder or due to an inflammatory, infectious, congenital or drug-induced cause.

The method of the invention is performed using an MR system. Reference is made to FIG. 5, which depicts an apparatus 1010 in which the systems and methods for performing the invention may be implemented. The apparatus 1010 may be embodied in any one of a wide variety of wired and/or wireless computing devices, multiprocessor computing device, and so forth. As shown in FIG. 5, the apparatus 1010 comprises memory 214, a processing device 202, a number of input/output interfaces 204, a network interface 206, a display 205, a peripheral interface 211, and mass storage 226, wherein each of these devices are connected across a local data bus 210. The apparatus 1010 may be coupled to one or more peripheral measurement devices (not shown) connected to the apparatus 1010 via the peripheral interface 211.

The processing device 202 may include any custom made or commercially-available processor, a central processing unit (CPU) or an auxiliary processor among several processors associated with the apparatus 1010, a semiconductor based microprocessor (in the form of a microchip), a macro-processor, one or more application specific integrated circuits (ASICs), a plurality of suitably configured digital logic gates, and other well-known electrical configurations comprising discrete elements both individually and in various combinations to coordinate the overall operation of the computing system.

The memory 214 can include any one of a combination of volatile memory elements (e.g. random-access memory (RAM, such as DRAM, and SRAM, etc.)) and non-volatile memory elements (e.g. ROM, hard drive, tape, DVD, etc.). The memory 214 typically comprises a native operating system 216, one or more native applications, emulation systems, or emulated applications for any of a variety of operating systems and/or emulated hardware platforms, emulated operating systems, etc. For example, the applications may include application specific software which may be configured to perform some or all of the systems and methods for producing images as described herein. In accordance with such embodiments, the application specific software is stored in memory 214 and executed by the processing device 202. One of ordinary skill in the art will appreciate that the memory 214 can, and typically will, comprise other components which have been omitted for purposes of brevity.

Input/output interfaces 204 provide any number of interfaces for the input and output of data. For example, where the apparatus 1010 comprises a personal computer, these components may interface with one or more user input devices 204. The display 205 may comprise a computer monitor, a plasma screen for a PC, a liquid crystal display (LCD) on a hand held device, or other display device.

In the context of this disclosure, a non-transitory computer-readable medium stores programs for use by or in connection with an instruction execution system, apparatus, or device. More specific examples of a computer-readable medium may include by way of example and without limitation: a portable computer diskette, a random access memory (RAM), a read-only memory (ROM), an erasable programmable read-only memory (EPROM, EEPROM, or Flash memory), and a portable compact disc read-only memory (CDROM) (optical).

With further reference to FIG. 5, network interface device 206 comprises various components used to transmit and/or receive data over a network environment. For example, the network interface 206 may include a device that can communicate with both inputs and outputs, for instance, a modulator/demodulator (e.g., a modem), wireless (e.g., radio frequency (RF)) transceiver, a telephonic interface, a bridge, a router, network card, etc.). The apparatus 1010 may communicate with one or more computing devices (not shown) via the network interface 206 over a network 118. The apparatus 1010 may further comprise mass storage 226. The peripheral 211 interface supports various interfaces including, but not limited to IEEE-1394 High Performance Serial Bus (Firewire), USB, a serial connection, and a parallel connection.

The apparatus 1010 shown in FIG. 5 may be embodied, for example, as a magnetic resonance apparatus, which includes a processing module or logic for performing conditional data processing, and may be implemented either off-line or directly in a magnetic resonance apparatus. For such embodiments, the apparatus 1010 may be implemented as a multi-channel, multi-coil system with advanced parallel image processing capabilities, and direct implementation makes it possible to generate immediate images available for viewing immediately after image acquisition, thereby allowing re-acquisition on-the-spot, if necessary.

The medical imaging device may be, for example, a magnetic resonance imaging (MRI) device or magnetic resonance (MR) scanner.

A subject, such as a human patient, may be positioned in association with the MRI device. A region of the subject, e.g. all or part of the tissue or organ, may be selected for imaging.

One or more of Bo shimming, centre frequency adjustments and trigger delay selection may be performed before imaging in order to reduce off-resonance and motion artefacts.

In Step (a), an MR data set from all or part of a tissue or organ of a subject is acquired. Generally, such a data set will be a k-space data set. K-space is the temporary image space in which data from digitized MR signals is stored during data acquisition and comprises raw data in a spatial frequency domain before reconstruction. When sufficient data to fill k-space (either by sampling directly the whole of k-space or through acceleration methods such as parallel imaging or compressed sensing) has been acquired (at the end of an MR scan), the data is mathematically processed to produce an image.

The MR data set is acquired using a pulse sequence, i.e. an MR pulse sequence. The first and second components may be the same or different readout types.

The aim of the first component of the pulse sequence is to provide T2-weighted or T2*-weighted MR data. Preferably, the first component of the pulse sequence provides strong T2-weighted or T2*-weighted MR data. As used herein, the term “strong” means that a change of 20% in the T2 or T2* from the tissue or organ, or part thereof, will lead to a change of at least 10% in the resulting pixel signal intensity.

Preferably, the first component of the pulse sequence provides T2-weighted or T2*-weighted fast readout. As used herein, the term “fast” relates to acquiring multiple k-space lines in each imaging readout and/or otherwise temporally efficiently sampling k-space with e.g. a spiral readout.

Examples of suitable first component readout types include a T2-preparation module (or T2* preparation module) followed by a gradient echo readout, e.g. RF-spoiled gradient echo (FLASH), steady state free precession (SSFP) or balanced SSFP (bSSFP); inherently T2-weighted readouts, e.g. single shot fast spin echo or spin echo EPI; or inherently T2*-weighted readouts, e.g. long echo time GRE/FLASH, GRE-EPI or FLASH. Examples of such components are well known in the art (e.g. Handbook of MRI Pulse Sequences, Matt A Bernstein, Kevin F King and Xiaohong Joe Zhou. Elsevier Academic Press, Burlington Mass. (2004)). Preferably, the first component is a T2 prepared bSSFP or FLASH. Most preferably, the first component is a T2-prepared segmented bSSFP sequence.

The second component is a low flip angle readout without additional magnetisation preparation. The aim of the second component of the pulse sequence is to provide a reference component. Intrinsically, it will be proton density weighted, but in practice it will have some T1 and T2 weighting due to the recovering magnetisation. Examples of suitable second component readout types include low flip-angle GRE, SPGR, FLASH and GRE-EPI. The second component must be a non-T2-prepared signal. In some embodiments, the second component is a fast readout.

Preferably, the second component comprises a low flip angle FLASH readout. Most preferably, the flip-angle is 1 to 10°, more preferably 3 to 5°.

In some preferred embodiments of the invention, the first component of the pulse sequence is segmented T2-prepared bSSFP and the second component of the pulse sequence is segmented 5° FLASH.

Hence the method of the invention comprises the step of acquiring MR signal data with first and second sequences as defined above.

Preferably, the pulse sequence is synchronized with the subject's ECG signal to acquire MR data during a rest phase of a subject's heart cycle, i.e. the pulse sequence is ECG-gated. This improves data accuracy by minimizing cardiac motion artefacts in the acquired data.

If the first component produces insufficient T2- or T2*-weighting natively, a magnetisation preparation module may be inserted to induce this weighting in the longitudinal magnetisation of the first component and to obtain a steady state. This is then sampled using the aforementioned readout. Magnetisation preparation may, for example, be achieved as in (25).

The second component does not comprise additional magnetisation preparation.

Preferably, a plurality of first and second component pulse sequence pairs are generated in order to achieve steady state in the MR system before the first MR data sets are acquired.

The pulse sequence comprises alternating first and second components. Second components of the pulse sequence are interleaved between the first components of the pulse sequence. The very first component (temporally) in the pulse sequence may be the first component or the second component.

Preferably, the pulse sequence comprises a plurality of first and second components, wherein one second component of the pulse sequence is interleaved between adjacent pairs of first components of the pulse sequence.

Second components of the pulse sequence are interleaved between all or substantially all of the first components of the pulse sequence.

In some embodiments, the second components of the pulse sequence are interleaved equidistantly between adjacent pairs of first components of the pulse sequence. In some embodiments, the second components of the pulse sequence are interleaved non-equidistantly between adjacent pairs of first components of the pulse sequence.

The first and/or second components of the pulse sequences are preferably temporally regularly spaced.

In some embodiments, first components are temporally regularly spaced, one second component is interleaved between adjacent pairs of first components, and the time interval between the second component and the subsequent first component is less than the time interval between the first component and the subsequent second component. In other embodiments, first components are temporally regularly spaced, one second component is interleaved between adjacent pairs of first components, and the time interval between the second component and the subsequent first component is greater than the time interval between the first component and the subsequent second component.

In some embodiments, the time interval between the first component and the subsequent second component is 0.1-10%, 10-20%, 20-30%, 30-40%, 40-50%, 50-60%, 60-70%, 70-80%, 80-90% or 90-99.9% of the total time interval between consecutive first components. Preferably, the time interval between the first component and the subsequent second component is 50-60%, 60-70%, 70-80%, 80-90% or 90-99.9% of the total time interval between consecutive first components, more preferably 80-85%, 85%-90%, 90-95% or 95-99.9% of the total time interval between consecutive first components (see FIG. 8 herein).

Examples of some pulse sequences which can be used in the method of the invention are known (e.g. Siemens WIP 657, VB17).

In some embodiments, the MR data set is preferably acquired at systole or mid-diastole. In other embodiments, the MR data set is not acquired at systole or is not acquired at mid-diastole. In yet other embodiments, no attempt is made to obtain the MR data set at a specified stage of the cardiac cycle.

Step (b) relates to generating at least two image datasets from the MR dataset, a first image dataset derived from the signals obtained from the first component of the pulse sequence, and a second image dataset derived from the signals obtained from the second component of the pulse sequence.

The image datasets represent individual reconstructed pixel signal intensities. Such images are generated using standard methods.

The images in the second dataset may be smoothed and/or de-noised prior to the normalisation process.

Step (c) relates to normalising the first image dataset using the second image dataset as a reference dataset to produce a third image dataset. The individual reconstructed pixel signal intensities in the images in the first dataset are divided by the individual reconstructed pixel signal intensities in the images in the second dataset to produce the third (normalised) image dataset. The individual reconstructed pixel signal intensities in the images in the first dataset may also be combined with the individual reconstructed pixel signal intensities in the images in the second dataset (to produce the third (i.e. normalised) image dataset) using other mathematical functions. This produces a heart-rate compensated signal intensity map (image) of all or part of the subject's tissue or organ. Inherently, this step will also normalise the third image dataset for the distance from any surface coil.

In Step (d), a heart-rate compensated MR image of all or part of the subject's tissue or organ is optionally displayed from the third image data set. In some embodiments, heart-rate compensated MR image is displayed on a visual display. Preferably, all or part of the heart-rate compensated MR image is displayed in colour (e.g. a colour map), wherein different signal intensity values or ranges are represented by different colours.

The flowchart of FIG. 6 shows examples of functionality that may be implemented in the apparatus of FIG. 5. Whilst FIG. 6 illustrates the invention with reference to a “heart/visceral organ of interest”, the invention should not be viewed as being limited in this way. The heart/visceral organ may be replaced by other tissues and organs, or parts thereof, as defined herein.

If embodied in software, each block shown in FIG. 5 may represent a module, segment, or portion of code that comprises program instructions to implement the specified logical function(s). The program instructions may be embodied in the form of source code that comprises machine code that comprises numerical instructions recognizable by a suitable execution system such as the processing device (FIG. 5) in a computer system or other system. The machine code may be converted from the source code, etc. If embodied in hardware, each block may represent a circuit or a number of interconnected circuits to implement the specified logical function(s).

Although the flowchart of FIG. 6 shows a specific order of execution, it is understood that the order of execution may differ from that which is depicted. For example, the order of execution of two or more blocks may be scrambled relative to the order shown. Also, two or more blocks shown in succession in FIG. 6 may be executed concurrently or with partial concurrence. Further, in some embodiments, one or more of the blocks shown in FIG. 6 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein that comprises software or code can be embodied in any non-transitory computer-readable medium for use by or in connection with an instruction execution system such as, for example, a processing device in a computer system or other system. In this sense, each may comprise, for example, statements including instructions and declarations that can be fetched from the computer-readable medium and executed by the instruction execution system.

In some embodiments of the invention, the method is not performed on subjects (preferably human subjects) under non-ambient CO₂ conditions, e.g. conditions such that the partial pressure of CO₂ was artificially raised (e.g. 1-10 mmHg) or reduced (e.g. 1-10 mmHg) compared to ambient partial pressures of CO₂. In particular, in some embodiments, the method is not performed on subjects (preferably human subjects) under hypercapnic conditions.

In yet another embodiment, there is provided a computer-implemented method of obtaining an indication of the differences in the performance of all or part of a subject's tissue or organ under different conditions, the method comprising the steps of:

(A) obtaining a first heart-rate-compensated magnetic resonance (MR) image of all or part of a tissue or organ of a subject, by a method of the invention, wherein the MR image is obtained whilst subjecting the subject or all or part of the subject's tissue or organ to a first set of conditions;

(B) obtaining a second heart-rate-compensated magnetic resonance (MR) image of all or part of the tissue or organ of the subject, by a method of the invention, wherein the MR image is obtained whilst subjecting the subject or all or part of the subject's tissue or organ to a second set of conditions, wherein the first set of conditions are different from the second set of conditions; and

(C) comparing the first and second MR images to obtain an indication of the differences in the performance of all or part of the subject's tissue or organ under the first and second conditions.

Preferably, the tissue or organ is a heart, most preferably a human heart.

Examples of such conditions include:
(i) a first set of conditions wherein the subject is under a stress; and
(ii) a second set of conditions where the subject is at rest.

Further examples of such conditions include:
(i) a first set of conditions where the subject is at rest but has been exercising for a prescribed period beforehand (e.g. 1-10 minutes); and
(ii) a second set of conditions wherein the subject is at rest and has been at rest for a prescribed period beforehand (e.g. 1-10 minutes).

Examples of other conditions include:
(i) a first set of conditions wherein a vasoactive agent has (recently) been administered to the subject; and
(ii) a second set of control conditions (wherein a vasoactive agent has not (recently) been administered to the subject).

Examples of vasoactive agents include vasodilatory agents (e.g. adenosine) and vasoconstrictive agents.

Examples of other conditions include:
(i) a first set of conditions wherein the subject is subjected to physiologically-tolerable hypercapnic conditions; and
(ii) a second set of control conditions wherein the subject is not subjected to physiologically-tolerable hypercapnic conditions.

Preferably, in these two sets of conditions, the method of the invention requires the subject to hold his/her breath during MR data acquisition (i.e. breath-holding conditions).

In some embodiments, the first and second images are displayed visually and the two images are compared visually, e.g. by eye. In other embodiments, the first and second images may be compared mathematically, and the differences between the two images (e.g. at a segmental level, pixel by pixel level, or voxel by voxel level) may be displayed.

For a given specific protocol and field strength, the normal limits for rest and stress can be used to set thresholds in the (colour) map used for display.

In some embodiments, the method comprises:
(C) normalising the third image dataset for the first MR image using the third image dataset for the second MR image as a reference dataset to produce a fourth image dataset; and optionally
(D) displaying, from the fourth image dataset, an image which represents a change in image intensity between the first and second sets of conditions.

In some embodiments, the comparison step may be useful in the diagnosis of a heart disorder in the subject, e.g. where tissue oxygenation determines either a change in metabolism or tissue perfusion is affected.

In a further embodiment, the invention provides a system or apparatus comprising at least one processing means arranged to carry out the steps of a method of the invention.

The processing means may, for example, be one or more computing devices and at least one application executable in the one or more computing devices. The at least one application may comprise logic to carry out the steps of a method of the invention.

In a further embodiment, the invention provides a carrier bearing software comprising instructions for configuring a processor to carry out the steps of a method of the invention.

The disclosure of each reference set forth herein is specifically incorporated herein by reference in its entirety.

### BRIEF DESCRIPTION OF THE FIGURES

Many aspects of the disclosure can be better understood with reference to the following Figures. The components in the Figures are not necessarily to scale, emphasis instead being placed upon clearly illustrating the principles of the present disclosure. Moreover, in the figures, like reference numerals designate corresponding parts throughout the several views.

FIG. 1. Comparison of mean signal intensity change of mBOLD and nBOLD showing the wide variance of mBOLD.

FIG. 2. Comparison of mean difference in segmental BOLD SIΔ for mBOLD (blue) and nBOLD (green) confirms a greater variance in segmental measurement of mBOLD. (Error Bars represent standard deviation).

FIG. 3. Two cases illustrating the difference in grey scale and colour for healthy subjects during rest (left) and stress (right).

FIG. 4. A case showing the easy identification of off-resonance artefacts (red arrow), which is more apparent on the colour map (B) as compared to the grey scale (A).

FIG. 5 is a schematic block diagram of an apparatus in which embodiments of the method of the invention may be implemented.

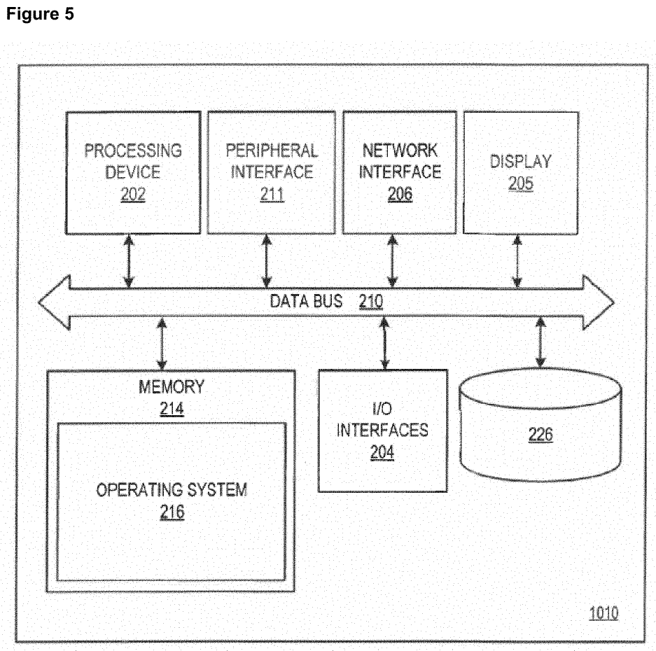

FIG. 6 shows a flowchart depicting some example methods of obtaining MRI images according to the invention.

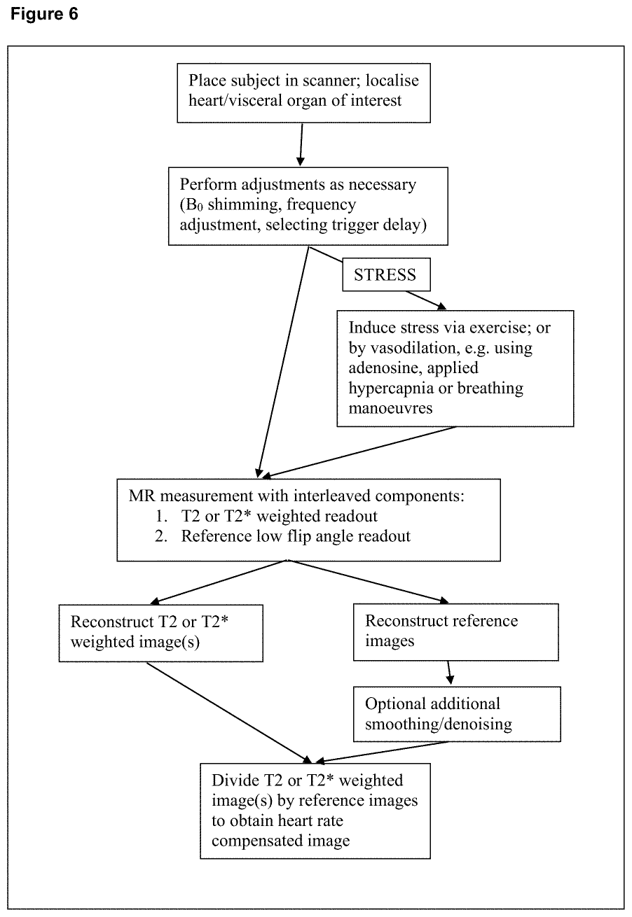

FIG. 7 shows a Bloch simulation of the longitudinal magnetisation during an experiment and includes the timings of the T2-prepared SSFP (first readout) and FLASH (second readout), with the SSFP and FLASH equidistant between the two.

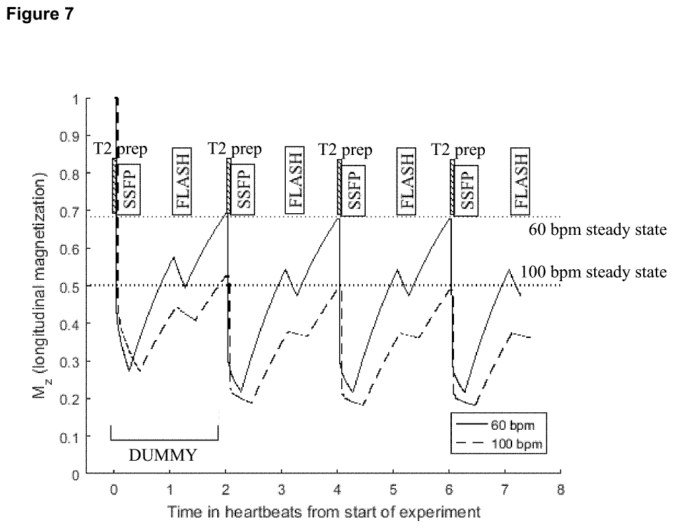

FIG. 8 shows the same as FIG. 7, but in the case that the second, FLASH readout is delayed to run immediately prior to the T2-prepared SSFP (first) readout.

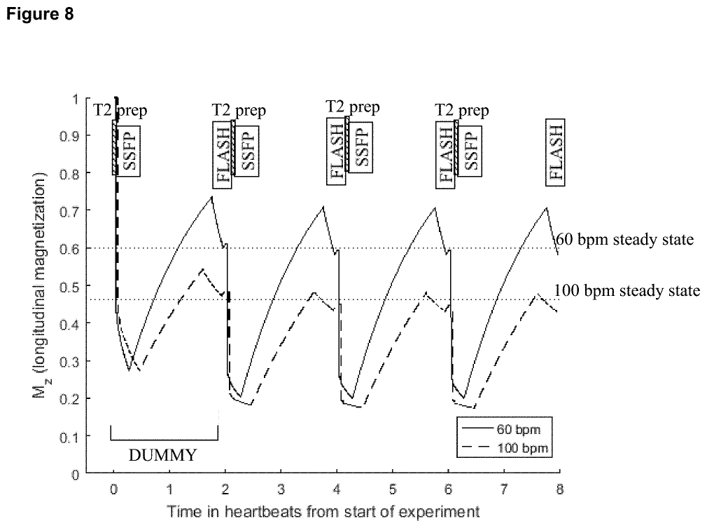

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

Thus, various modifications of the invention in addition to those shown and described herein will be apparent to those skilled in the art from the foregoing description. Such modifications are also intended to fall within the scope of the appended claims.

### Example 1: Methods

Bloch Simulations Bloch simulations were carried out in order to modify the heart rate correction previously reported (12) to account for the additional FLASH readout and heartbeat between SSFP readouts. The T2 prep module was modelled as a multiplication in longitudinal magnetization, M_{z}, during a time TEₚᵣₑₚ, by a factor exp(-T2/TEₚᵣₑₚ), where TEₚᵣₑₚ is the T2 prep echo time of 40 ms. The SSFP and FLASH readouts were implemented with identical timing to the imaging sequence, with TR/TE=2.86 ms/1.43 ms, 72 readout lines per heartbeat, flip angles of 44° (SSFP) and 5° (FLASH), with 10 linear ramp up pulses for SSFP. The two images were acquired in an interleaved fashion over three heartbeats each (six in total) with dummy SSFP and FLASH acquisitions beforehand (eight heartbeats total). In order to represent RF spoiling in the FLASH readout, M_{xy} was reset to zero at the end of each short TR period. The mean M_{z} just prior to each T2 prep was averaged to determine the steady state longitudinal magnetization. Myocardial T1 was set at 1471 ms and T2 at 44 ms to represent normal values at 3T (14). The sequence was simulated at RR intervals from 400 ms to 1500 ms in 50 ms increments.

An exponential of the form

M_{z}=1−βe^{-RR/T}^{_{1,mod}}

was fitted to the resulting steady-state M_{z} to produce an expression for heart rate correction in the same form as used in previous work (6, 12).

Population

CMR data from twenty healthy subjects was retrospectively analysed to address the aims of this study. Subjects had previously been scanned in a study was approved by the institutional ethics committee (reference12/LO/1979) and were selected as the first subjects in the study with SSFP BOLD imaging free of susceptibility artefacts. All subjects were regarded as healthy with no previous medical history, cardiac disease or risk factors for cardiac disease.

CMR Protocol

All 20 participants underwent cardiac magnetic resonance (CMR) at 3 Tesla (3T), Trio MR scanner (Siemens, Erlangen, Germany) for cine, adenosine stress BOLD and perfusion imaging. Participants were instructed to refrain from caffeine-containing drinks and food for at least 24 hours preceding the study. Cine CMR was planned and acquired from standard pilot images. Short-axis cine images covering the entire left ventricle were acquired using a retrospectively ECG-gated SSFP sequence (echo time, 1.5 ms; repetition time, 3 ms; flip angle, 50°). For BOLD-CMR, a single basal slice was acquired at systole using an ECG-gated T2-prepared segmented SSFP sequence with interleaved low flip angle FLASH reference images (13) (Siemens WIP 567, VB17). The sequence parameters matched those used for the Bloch simulations. This sequence outputs two images, the SSFP image alone, labelled “mag”, and the SSFP divided by the interleaved FLASH image, labelled “norm”. We use “magnitude” and “normalized” herein to refer to these images and signal intensities derived from them. Shimming and centre frequency adjustments were performed before BOLD imaging to generate images free from off-resonance artefacts. Adenosine was then infused at a dose of 140 mcg/kg/min and at peak vasodilator stress (at least 3-4 minutes) a slice-matched stress BOLD image was acquired. Blood pressure was recorded by a vital signs monitor machine at baseline and at 1-minute intervals during stress. Following the acquisition of stress BOLD images, first pass perfusion imaging was undertaken using a T1-weighted gradient echo sequence with saturation recovery magnetization preparation. A dose of 0.03 mmol/kg of Gadoterate Meglumine was injected at 6 ml/sec during stress followed by a saline flush 12 ml at 6 ml/sec and the same dose for rest acquisition (15).

CMR Image Analysis

Commercially available software (Circle Cardiovascular Imaging Inc., Calgary, Canada) was used to analyse left ventricular (LV) volumes, mass, ejection fraction (16, 17), myocardial perfusion reserve index (MPRI) and BOLD SI. Quantitative analysis of rest and stress BOLD images without (magnitude image; mBOLD SI) and with FLASH normalisation (normalised image; nBOLD SI) were undertaken by two observers (MH and KC). The signal intensity in the magnitude images was HR corrected based on the Bloch simulations described above. BOLD ΔSI was estimated as the relative increase in signal intensity between rest and stress BOLD images as previously described (12). For perfusion analysis, signal intensity curves were generated to measure MPRI as previously described (18).

To assess intra-observer variability, measurements were repeated on both magnitude and normalized imaged for the same subjects by one of the observers (KC) after two weeks.

Commercially available software (Circle Cardiovascular Imaging Inc., Calgary, Canada) T2 mapping module was also used to develop a colour map to visually represent SI variations in the myocardium based on the signal intensity ranges in the normalized images. Bright green was used to represent pixels with SI two standard deviations (2 SD) lower than the mean rest BOLD SI (˜200 AU) and orange for SI 2 SD above the mean rest SI˜238 AU. Coincidentally, this SI was also 2 SD below the mean segmental stress SI. Finally, red was used for the highest signal intensity ˜280 AU (2SD above the mean stress SI). For SI below the physiological range (˜175 AU), we used blue.

Statistical Analysis

All statistical analyses were undertaken using IBM SPSS Statistics version 23.0 (IBM Corp., Armonk, N.Y., USA), except for the tests for normality and linear mixed modelling which were carried out in Matlab (Mathworks, Natick, Mass.). Analysis was carried out for slice-averaged data for the raw signal intensities in the normalized and HR-corrected magnitude images, and for both slice-averaged and segmental nBOLD and mBOLD signals. All variables were tested for normality with the Kolmogorov-Smirnov test with p>0.1 (for normality tests only) taken to indicate data consistent with a normal distribution.

Data (slice/segmentally averaged, signal intensities and BOLD ΔSI) were characterized by mean and standard deviation and the coefficient of variation calculated. A one-sided F-test was used to test whether the population variance was reduced in slice-averaged SI from normalized images relative to that from magnitude images.

Paired, two-sided t-tests were used to test whether nBOLD and mBOLD were statistically significantly different from each other, both for the whole slice and for each segment, and f-tests used to test whether both whole slice and segmental nBOLD variance was lower than mBOLD variance. Linear mixed models were used to assess the dependence of segmental mBOLD, nBOLD and BOLD difference (mBOLD-nBOLD) on the fixed effects segment, rest HR, stress HR, and segmental MPRI. Subject intercept was included as a random parameter and models were compared using likelihood ratio tests to determine whether the inclusion of the fixed effects one by one improved the model and should therefore be included. Visual inspection of residual plots did not reveal any obvious deviations from homoscedasticity or normality. Statistical significance was indicated by p<0.05.

Two-way random Intra Class Correlation (ICC) was used to assess the level of agreement between observers and two-way mixed ICC was used to level of agreement within the same observer at a per-segment level and per-subject level. Reproducibility was deemed to have improved statistically significantly if the confidence intervals did not overlap.

### Example 2: Results

Bloch Simulations

The resulting equation for HR correction of magnitude images was

$$S = \frac{{S}_{0}}{1 - 0.98 {e}^{- RR / 980}} \qquad ( 1 )$$

where S₀ is the measured signal intensity, S is the heart rate corrected signal intensity, and RR denotes the RR interval during the BOLD acquisition in ms.

Baseline Characteristics

Data from all 20 subjects and all 240 (rest and stress) segments were included for the analysis. Baseline characteristics are listed in Table 1. Mean age of all subjects was 47±15 years. Eleven (55%) out of 20 were male. Left ventricular indices and myocardial perfusion reserve indexes were within normal limits. All patients had a low (<10%) 10 year Framingham risk of coronary disease. Signal intensities and BOLD ΔSI, both whole slice and segmental, as well as heart rate changes, were all normally distributed.

TABLE 1
Baseline characteristics of healthy controls.

| | CMR (n = 20) |
|---|---|
| Age (years) | 42 ± 12 |
| Male, % (n) | 55 (11) |
| Body mass index (kg/m²) | 25 ± 3 |
| Rest heart rate (bpm) | 62 ± 13 |
| Stress heart rate (bpm) | 93 ± 20 |
| Absolute increase in heart rate | 31 ± 11 |
| Relative increase in heart rate | 50 ± 16% |

CMR findings

| | |
|---|---|
| LVEF, % | 63 ± 16 |
| LVEDV (ml) | 151 ± 31 |
| LVESV (ml) | 98 ± 10 |
| Stroke volume (ml) | 103 ± 21 |
| LV Mass (g) | 91 ± 15 |
| LV Mass index (g/m²) | 81 ± 28 |
| MPRI | 1.96 ± 0.38 |

Data are mean±standard deviation. LV, Left ventricular; EDV, end-diastolic volume; ESV, end-systolic volume; EF, ejection fraction; MPRI Myocardial perfusion reserve index, bpm beats per minute.

FLASH-“normalized” and HR-corrected “magnitude” image signal intensities

Slice Level Comparisons

In the mean heart rate (HR) corrected mBOLD SI mean and (HR uncorrected) nBOLD SI at rest and stress, an F-test showed that the variance in SI was statistically significantly reduced in the nBOLD images at both rest and stress (p<0.0001).

mBOLD and nBOLD

Slice Comparisons

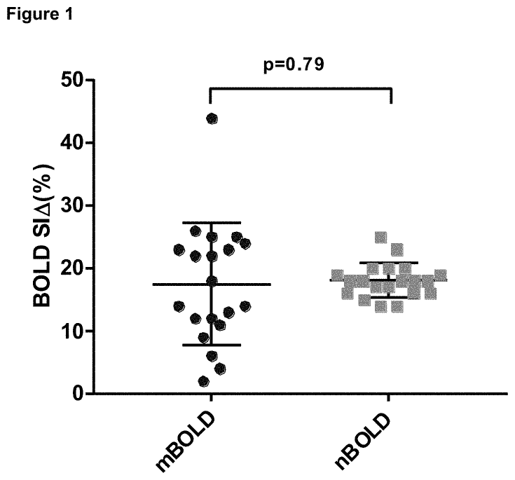

The relative increase in SI for mBOLD and nBOLD during stress were similar 17±10% and 18±3% respectively, with no statistically significant difference between the two (p=0.79) (FIG. 1), corresponding to coefficients of variance of 59% and 17%. mBOLD ΔSI has a significantly higher variance compared to nBOLD ΔSI on a per-slice/subject basis (p<0.0001).

Segmental Comparisons

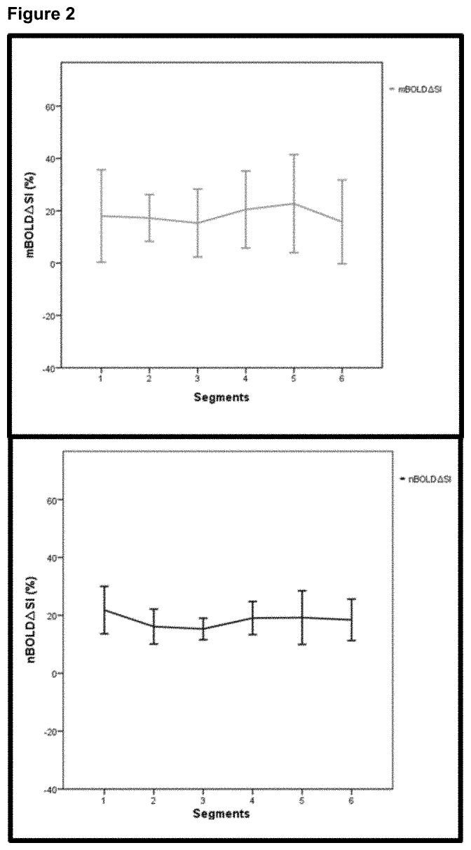

Segmental mBOLD and nBOLD ΔSI are shown in Table 2, along with the results of the statistical comparisons of values and variances. There was no significant difference in BOLD values between mBOLD and nBOLD, and but the AS segment showed a statistically significant improvement in variance with nBOLD over mBOLD. The data are also presented in FIG. 2.

TABLE 2
Comparison of segmental ΔSI for mBOLD and nBOLD images.

| | | | | | Comparison p-values | |
|---|---|---|---|---|---|---|
| | | | | | t-test | f-test |
| | mBOLD ΔSI | | nBOLD ΔSI | | (difference | (difference |
| | Mean ± s.d. | CoV | Mean ± s.d. | CoV | in means) | in variances) |
| Slice | 17.4% ± 9.8% | 56% | 18.1% ± 2.8% | 15.5% | 0.75 | <0.001 |
| average | | | | | | |
| A | 18.0% ± 17.7% | 98% | 21.8% ± 8.2% | 38% | 0.280 | 0.001 |
| AS | 17.2% ± 8.9% | 52% | 16.1% ± 6.05% | 38% | 0.567 | 0.09 |
| IS | 15.3% ± 13.0% | 85% | 15.3% ± 3.7% | 24% | 0.994 | <0.001 |
| I | 20.5% ± 14.7% | 70% | 19.1% ± 5.7% | 30% | 0.691 | <0.001 |
| IL | 22.7% ± 18.7% | 82% | 19.2% ± 9.3% | 48% | 0.346 | 0.003 |
| AL | 15.8% ± 16.0% | 101% | 18.5% ± 7.2% | 39% | 0.426 | <0.001 |

A anterior, AS anteroseptal, IS Inferoseptal, I Inferior, IL Inferolateral, AL Anterolateral

Origins of Differences Between mBOLD and nBOLD

Building linear mixed models for segmental BOLD responses showed that mBOLD ΔSI only showed a statistically significant dependence on stress heart rate (0.23%/bpm, equivalent to 17% BOLD ΔSI for the range of stress heart rates in these normal volunteers, p=0.03). In contrast, nBOLD ΔSI had no dependence on heart rate, rest or stress, but did have some segmental dependence (anterior ΔSI was 6.5% higher than inferoseptal, p=0.003). Only the heart rate dependence of mBOLD was reflected in the mixed model of the BOLD difference (mBOLD-nBOLD), which had a similar dependence on stress HR (0.24%/bpm, p=0.04) but no segmental dependence.

Slice and Segmental Reproducibility

On a slice-level, inter- and intra-observer ICC for nBOLD were excellent at 0.88 (95% CI 0.71-0.95) and 0.90 (95% CI 0.74-0.96), p<0.001. Similarly, mBOLD had a high inter-observer ICC and intra-observer ICC at 0.84 (95% CI 0.59-0.93) and 0.92 (95% CI 0.79-0.97), p<0.001 respectively.

On a segmental level, nBOLD had a higher inter- and intra-observer ICC compared to mBOLD with very minimal overlap of confidence intervals (Table 3).

TABLE 3
Inter-observer and intra-observer intra-class correlation
coefficient for segmental analysis for mBOLD and nBOLD

| | ICC | 95% CI | p-value |
|---|---|---|---|
| mBOLD | | | |
| Interobserver | 0.77 | 0.67-0.84 | <0.0001 |
| Intraobserver | 0.85 | 0.76-0.90 | <0.0001 |
| nBOLD | | | |
| Interobserver | 0.89 | 0.84-0.92 | <0.0001 |
| Intraobserver | 0.92 | 0.89-0.95 | <0.0001 |

CI = confidence interval; ICC = intraclass correlation.

Colour Map

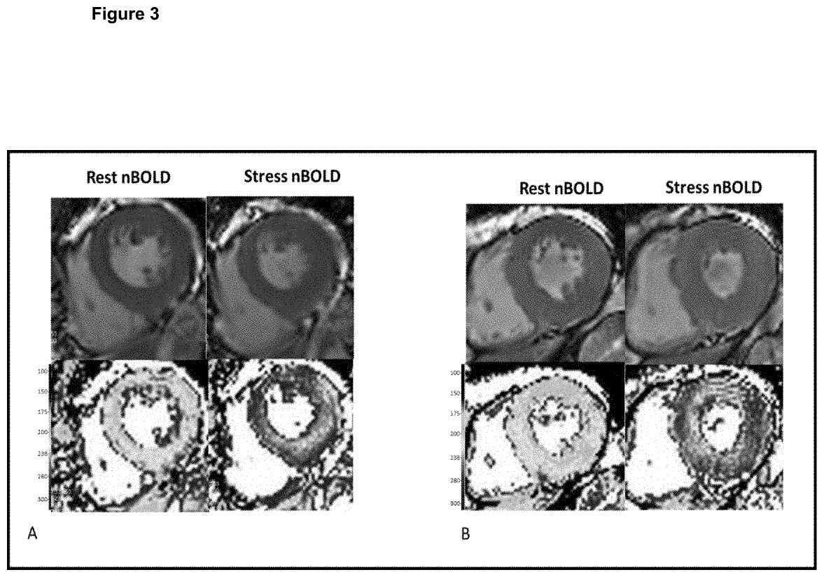

Two examples of applying the standardized colour map derived from the normal population limits in the normalized rest and stress signal intensities are shown in FIG. 3. When applied to the normalized images, the colour map enables a clear visualization of the difference between rest and stress perfusion in this group of normal volunteers, even before calculating the change in BOLD SI.

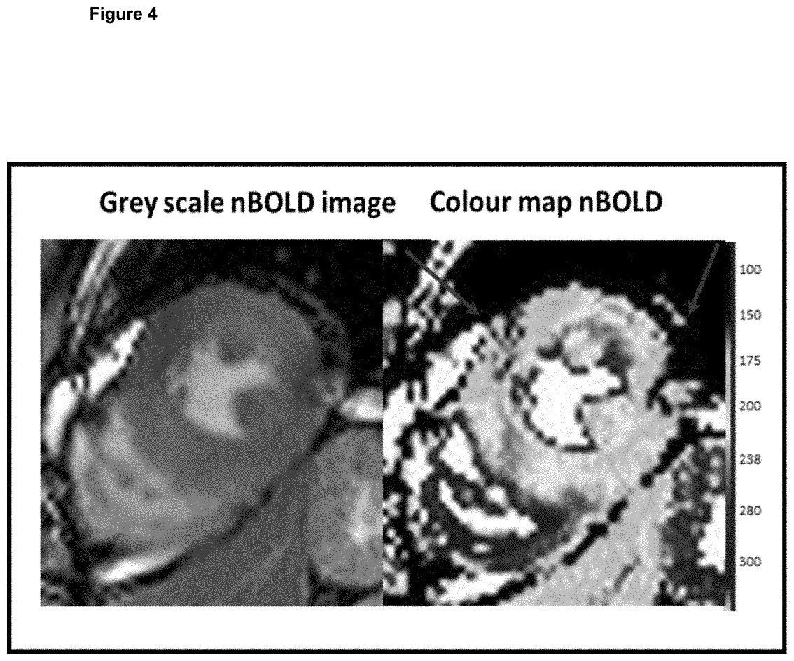

The application of the colour map to the normalised image without the need for additional HR correction also enabled the rapid identification of artefacts which are otherwise difficult to appreciate on the grey scale magnitude image. FIG. 4 demonstrates an example of susceptibility artifact near the heart lung interface which can be more clearly visualized using a colour map than in the grey scale image.

### REFERENCES

1. Ogawa S, Lee T M, Kay A R, Tank D W. Brain magnetic resonance imaging with contrast dependent on blood oxygenation. Proc Natl Acad Sci USA. 1990; 87(24):9868-72.
2. Atalay M K, Forder J R, Chacko V P, Kawamoto S, Zerhouni E A. Oxygenation in the rabbit myocardium: assessment with susceptibility-dependent MR imaging. Radiology. 1993; 189(3):759-64.
3. Wendland M F, Saeed M, Lauerma K, de Crespigny A, Moseley M E, Higgins C B. Endogenous susceptibility contrast in myocardium during apnea measured using gradient recalled echo planar imaging. Magn Reson Med. 1993; 29(2):273-6.
4. Walcher T, Manzke R, Hombach V, Rottbauer W, Wohrle J, Bernhardt P. Myocardial perfusion reserve assessed by T2-prepared steady-state free precession blood oxygen level-dependent magnetic resonance imaging in comparison to fractional flow reserve. Circ Cardiovasc Imaging. 2012; 5(5):580-6.
5. Vohringer M, Flewitt J A, Green J D, Dharmakumar R, Wang J, Jr., Tyberg J V, et al. Oxygenation-sensitive CMR for assessing vasodilator-induced changes of myocardial oxygenation. J Cardiovasc Magn Reson. 2010; 12:20.
6. Friedrich M G, Niendorf T, Schulz-Menger J, Gross C M, Dietz R. Blood oxygen level-dependent magnetic resonance imaging in patients with stress-induced angina. Circulation. 2003; 108(18):2219-23.
7. Wacker C M, Bock M, Hartlep A W, Bauer W R, van Kaick G, Pfleger S, et al. BOLD-MRI in ten patients with coronary artery disease: evidence for imaging of capillary recruitment in myocardium supplied by the stenotic artery. MAGMA. 1999; 8(1):48-54.
8. Fieno D S, Shea S M, Li Y, Harris K R, Finn J P, Li D. Myocardial perfusion imaging based on the blood oxygen level-dependent effect using T2-prepared steady-state free-precession magnetic resonance imaging. Circulation. 2004; 110(10):1284-90.
9. Fischer K, Guensch D P, Friedrich M G. Response of myocardial oxygenation to breathing manoeuvres and adenosine infusion. Eur Heart J Cardiovasc Imaging. 2015; 16(4):395-401.
10. Arnold J R, Karamitsos T D, Bhamra-Ariza P, Francis J M, Searle N, Robson M D, et al. Myocardial oxygenation in coronary artery disease: insights from blood oxygen level-dependent magnetic resonance imaging at 3 tesla. J Am Coll Cardiol. 2012; 59(22):1954-64.
11. Dass S, Cochlin L E, Suttie J J, Holloway C J, Rider O J, Carden L, et al. Exacerbation of cardiac energetic impairment during exercise in hypertrophic cardiomyopathy: a potential mechanism for diastolic dysfunction. European heart journal. 2015.
12. Karamitsos T D, Leccisotti L, Arnold J R, Recio-Mayoral A, Bhamra-Ariza P, Howells R K, et al. Relationship between regional myocardial oxygenation and perfusion in patients with coronary artery disease: insights from cardiovascular magnetic resonance and positron emission tomography. Circ Cardiovasc Imaging. 2010; 3(1):32-40.
13. Kellman P, Aletras A H, Mancini C, McVeigh E R, Arai A E. T2-prepared SSFP improves diagnostic confidence in edema imaging in acute myocardial infarction compared to turbo spin echo. Magn Reson Med. 2007; 57(5):891-7.
14. Stanisz G J, Odrobina E E, Pun J, Escaravage M, Graham S J, Bronskill M J, et al. T1, T2 relaxation and magnetization transfer in tissue at 3T. Magn Reson Med. 2005; 54(3):507-12.
15. Dass S, Cochlin L E, Suttie J J, Holloway C J, Rider O J, Carden L, et al. Exacerbation of cardiac energetic impairment during exercise in hypertrophic cardiomyopathy: a potential mechanism for diastolic dysfunction. European heart journal. 2015; 36(24):1547-54.
16. Karamitsos T D, Hudsmith L E, Selvanayagam J B, Neubauer S, Francis J M. Operator induced variability in left ventricular measurements with cardiovascular magnetic resonance is improved after training. J Cardiovasc Magn Reson. 2007; 9(5):777-83.
17. Li R, Yang Z G, Xu H Y, Shi K, Liu X, Diao K Y, et al. Myocardial Deformation in Cardiac Amyloid Light-chain Amyloidosis: Assessed with 3T Cardiovascular Magnetic Resonance Feature Tracking. Sci Rep. 2017; 7(1):3794.
18. Karamitsos T D, Dass S, Suttie J, Sever E, Birks J, Holloway C J, et al. Blunted myocardial oxygenation response during vasodilator stress in patients with hypertrophic cardiomyopathy. J Am Coll Cardiol. 2013; 61(11):1169-76.
19. Kellman P, Aletras A H, Mancini C, McVeigh E R, Arai A E. T2-prepared SSFP improves diagnostic confidence in edema imaging in acute myocardial infarction compared to turbo spin echo. Magn Reson Med. 2007; 57.21. Sung K, Nayak K S. Design and use of tailored hard-pulse trains for uniformed saturation of myocardium at 3 Tesla. Magn Reson Med. 2008; 60.
22. Noeske R, Seifert F, Rhein K H, Rinneberg H. Human cardiac imaging at 3 T using phased array coils. Magn Reson Med. 2000; 44(6):978-82.
23. Parnham S, Gleadle J M, Bangalore S, Grover S, Perry R, Woodman R J, et al. Impaired Myocardial Oxygenation Response to Stress in Patients With Chronic Kidney Disease. J Am Heart Assoc. 2015; 4(8):e002249.
24. Yang H J, Yumul R, Tang R, Cokic I, Klein M, Kali A, et al. Assessment of myocardial reactivity to controlled hypercapnia with free-breathing T2-prepared cardiac blood oxygen level-dependent MR imaging. Radiology. 2014; 272(2):397-406.
25. Brittain, J. H. et al., (1995), Coronary Angiography with Magnetization—Prepared T2 Contrast. Magn. Reson. Med., 33: 689-696).

1-20. (canceled)

21. A computer-implemented method for obtaining an indication of the differences in the performance of all or part a subject's tissue or organ under different conditions, the method comprising the steps of:
(A) obtaining a first heart-rate-compensated magnetic resonance (MR) image of all or part of a tissue or organ of a subject, by a method comprising the steps:
(a) acquiring, with an MR system, an MR data set from all or part of the tissue or organ of the subject using a pulse sequence, wherein the pulse sequence comprises at least two interleaved components:
(i) a first component, wherein the first component consists of a T2- or T2*-weighted readout, and
(ii) a second component, wherein the second component is a low flip angle readout without additional magnetisation preparation;
(b) generating at least two image datasets from the MR dataset:
a first image dataset derived from the signals obtained from the first component of the pulse sequence, and
a second image dataset derived from the signals obtained from the second component of the pulse sequence;
(c) normalising the first image dataset using the second image dataset as a reference dataset to produce a heart-rate-compensated MR image of all or part of the tissue or organ;
wherein this first heart-rate-compensated MR image is obtained whilst subjecting the subject or all or part of the subject's tissue or organ to a first set of conditions;
(B) obtaining a second heart-rate-compensated magnetic resonance (MR) image
of all or part of the tissue or organ of the subject by the method as defined in Step (A), (a)-(c), wherein this second heart-rate-compensated MR image is obtained whilst subjecting the subject or all or part of the subject's tissue or organ to a second set of conditions, wherein the first set of conditions are different from the second set of conditions; and
(C) comparing the first and second heart-rate-compensated MR images to obtain an indication of the differences in the performance of all or part of the subject's tissue or organ under the first and second conditions.

22. The method as claimed in claim 21, wherein the organ is a visceral organ; or a heart, liver, spleen, kidney, prostate, lung or pancreas.

23. The method as claimed in claim 21, wherein the tissue or organ is impaired or diseased.

24. The method as claimed in claim 21, wherein:
(A) the first component of the pulse sequence provides strong T2-weighted or T2*-weighted MR data; or
(B) the first component of the pulse sequence comprises a T2-preparation module or T2* preparation module.

25. The method as claimed in claim 21, wherein the first component of the pulse sequence comprises or consists of a gradient echo readout; an inherently T2-weighted readout; or an inherently T2*-weighted readout.

26. The method as claimed in claim 25, wherein the first component of the pulse sequence comprises or consists of a RF-spoiled gradient echo (FLASH), steady state free precession (SSFP) or balanced SSFP (bSSFP); a single shot fast spin echo or spin echo EPI; or a long echo time GRE/FLASH, GRE-EPI or FLASH.

27. The method as claimed in claim 21, wherein the first component of the pulse sequence comprises or consists of a T2 prepared bSSFP or FLASH, or a T2-prepared segmented bSSFP sequence.

28. The method as claimed in claim 21, wherein the second component of the pulse sequence comprises or consists of a low flip-angle GRE, SPGR, FLASH or GRE-EPI.

29. The method as claimed in claim 28, wherein the second component of the pulse sequence comprises or consists of a low flip angle FLASH readout, or a FLASH readout wherein the flip-angle is 1 to 10°, or 3 to 5°.

30. The method as claimed in claim 21, wherein the first component of the pulse sequence consists of a segmented T2-prepared bSSFP sequence, optionally with a T2-preparation module or T2* preparation module; and the second component of the pulse sequence consists of a segmented 5° FLASH sequence.

31. The method as claimed in claim 21, wherein the pulse sequence is synchronized with the subject's ECG signal to acquire MR data during a rest phase of the subject's heart cycle.

32. The method as claimed in claim 21, wherein the pulse sequence comprises a plurality of first and second components, wherein one second component of the pulse sequence is interleaved between adjacent pairs of first components of the pulse sequence.

33. The method as claimed in claim 32, wherein:
(A) the second components of the pulse sequence are interleaved equidistantly between adjacent pairs of first components of the pulse sequence; or
(B) the second components of the pulse sequence are interleaved non-equidistantly between adjacent pairs of first components of the pulse sequence.

34. The method as claimed in claim 33, wherein the first and second components are each temporally regularly spaced, one second component is interleaved between adjacent pairs of first components, and the time interval between the second component and the subsequent first component is less than the time interval between the first component and the subsequent second component.

35. The method as claimed in claim 34, wherein the time interval between the first component and the subsequent second component is 50-60%, 60-70%, 70-80%, 80-90% or 90-99.9% of the total time interval between consecutive first components, or 80-85%, 85%-90%, 90-95% or 95-99.9% of the total time interval between consecutive first components.

36. The method as claimed in claim 21, wherein a heart-rate compensated MR image of all or part of the subject's tissue or organ is displayed from the third image data set in colour wherein different signal intensity values or ranges are represented by different colours.

37. The method as claimed in claim 21, wherein:
(A) (i) the first set of conditions are wherein the subject is under a stress; and
(ii) the second set of conditions are wherein the subject is at rest; or
(B) (i) the first set of conditions are wherein the subject is at rest but has been exercising for a prescribed period beforehand; and
(ii) the second set of conditions are wherein the subject is at rest and has been at rest for a prescribed period beforehand.

38. The method as claimed in claim 21, wherein:
(A) (i) the first set of conditions are wherein a vasoactive agent has been administered to the subject; and
(ii) the second set of conditions are control conditions wherein a vasoactive agent has not been administered to the subject.

39. A system or apparatus comprising at least one processing means arranged to carry out the steps of the method as claimed in claim 21.

40. A carrier bearing software comprising instructions for configuring a processor to carry out the steps of the method as claimed in claim 21.
